(19) United States
(12) Patent Application Publication
(10) Pub. No.: US 2024/0272070 A1
(43) Pub. Date: Aug. 15, 2024

(54) INFORMATION PROCESSING DEVICE, INFORMATION PROCESSING METHOD, AND PROGRAM

(71) Applicant: SONY GROUP CORPORATION, TOKYO (JP)

(72) Inventors: DAIZO SHIGA, TOKYO (JP); TADAYOSHI MURAKAMI, TOKYO (JP); JUNKI INOUE, TOKYO (JP); YUKI SHIMIZU, TOKYO (JP)

(21) Appl. No.: 18/292,408

(22) PCT Filed: Mar. 22, 2022
(86) PCT No.: PCT/JP2022/012997

§ 371 Date: Jan. 26, 2024

(30) Foreign Application Priority Data
Aug. 10, 2021 (JP) 2021-130833

(51) Int. Cl. G01N 21/3554; F24C 7/08; G01N 33/02; G06T 7/00; G06V 40/20

(52) U.S. Cl. CPC G01N 21/3554; F24C 7/08; G01N 33/02; G06T 7/0004; G06V 40/20; G06T 2207/30128

(57) ABSTRACT

An information processing device (IP) includes a processor unit (PR). The processor unit (PR) detects a moisture amount of a cut ingredient (CI) on the basis of a sensing result of a cross section of the cut ingredient (CI) cut in accordance with progress of cooking. The processor unit (PR) calculates heating cooking time and a heating temperature of the cut ingredient (CI) on the basis of the moisture amount of the cut ingredient (CI).

### FIELD

The present invention relates to an information processing device, an information processing method, and a program.

### BACKGROUND

A cooking support system that supports cooking on the basis of sensor information has been known. For example, in a cooking support system of Patent Literature 1, time for heating cooking is automatically controlled on the basis of a size of an ingredient detected by a sensor.

### CITATION LIST

### Patent Literature

Patent Literature 1: Japanese Patent Application Laid-open No. 2020-166557

### SUMMARY

### Technical Problem

Even when cooking is performed according to a recipe, when a state of an ingredient to be used is different, finish of a dish is also different. It is not possible to handle a fresh ingredient and an ingredient, which is not sufficiently ripe or is dry, in the same manner. In a conventional cooking support system, since an internal state of the ingredient is not considered, the finish varies.

In general, it is difficult to estimate the internal state of the ingredient from an appearance. Conventionally, a cook acquires information such as an appearance and hardness of a cross section at a stage of cutting an ingredient, and changes preparation and a cooking method. In order to correct a cooking method for an ingredient having a large individual difference, experience knowledge as a cook is required. Thus, it is difficult for an inexperienced cook or an armature cook to reproduce finish of a dish.

Thus, the present disclosure proposes an information processing device, an information processing method, and a program capable of increasing a degree of reproduction of finish of a dish.

### Solution to Problem

According to the present disclosure, an information processing device is provided that comprises a processor unit that detects a moisture amount of a cut ingredient on a basis of a sensing result of a cross section of the cut ingredient cut in accordance with progress of cooking, and calculates heating cooking time and a heating temperature of the cut ingredient on a basis of the moisture amount of the cut ingredient. According to the present disclosure, an information processing method in which an information process of the information processing device is executed by a computer, and a program for causing the computer to execute the information process of the information processing device, are provided.

### BRIEF DESCRIPTION OF DRAWINGS

FIG. 1 is a view illustrating an outline of a cooking support system.

FIG. 2 is a view illustrating an example of a cooking scene.

FIG. 3 is a functional block diagram of an information processing device.

FIG. 4 is a view illustrating an example of a method of measuring a size of a cut ingredient.

FIG. 5 is a view illustrating an example of a method of measuring a moisture amount of the cut ingredient.

FIG. 6 is a view illustrating an example of gesture operation using an image sensor.

FIG. 7 is a view illustrating an example of a processing flow performed by the information processing device.

FIG. 8 is a view illustrating an example of a modification procedure of a reference recipe in a case where there is an additional foodstuff.

FIG. 9 is a view illustrating a flow of measurement work by a sensor unit.

FIG. 10 is a view illustrating a hardware configuration example of the information processing device.

### DESCRIPTION OF EMBODIMENTS

In the following, embodiments of the present disclosure will be described in detail on the basis of the drawings. In each of the following embodiments, overlapped description is omitted by assignment of the same reference sign to the same parts.

Note that the description will be made in the following order.
[1. Configuration of a cooking support system]
[2. Method of measuring a size of a cut ingredient]
[3. Method of measuring a moisture amount of a cut ingredient]
[4. Detection of gesture]
[5. Processing flow]
[5-1. Specification of reference data]
[5-2. Data acquisition for correction processing]
[5-3. Acquisition of data of a cut ingredient]
[5-4. Display of a cooking procedure]
[6. Hardware configuration example]
[7. Modification example]
[7-1. Addition of a weight sensor, a hardness sensor, and a temperature sensor]
[7-2. Transfer of cooking process data to a cooking equipment]
[8. Effect]

### 1. Configuration of a Cooking Support System

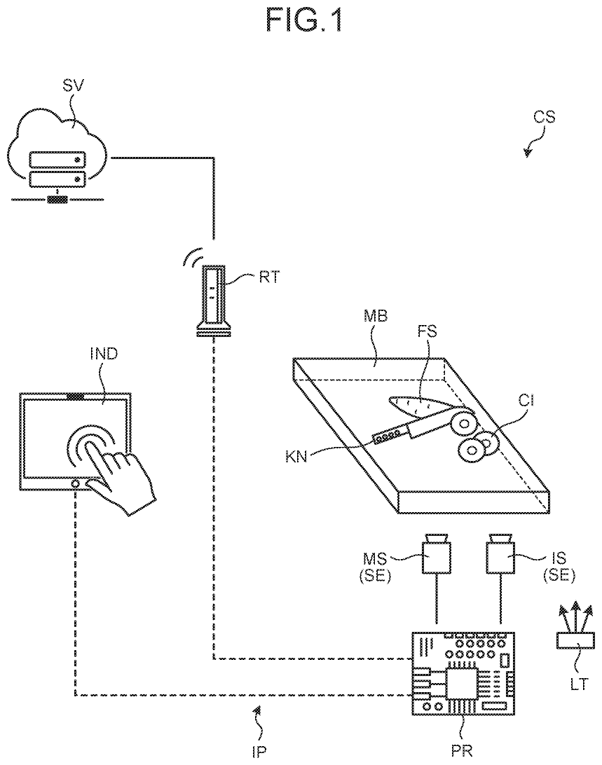

FIG. 1 is a view illustrating an outline of a cooking support system CSP.

The cooking support system CSP is a type of smart kitchen that supports cooking work by cooperation between a cooking equipment with a built-in sensor and an information terminal. For example, the cooking support system CSP supports heating cooking of a cut foodstuff FS (cut ingredient CI). Cooking support by the cooking support system CSP is performed by an information processing device IP. Although a solid line indicates connection by wired communication and a dotted line indicates connection by wireless communication in FIG. 1, the communication method is not limited thereto.

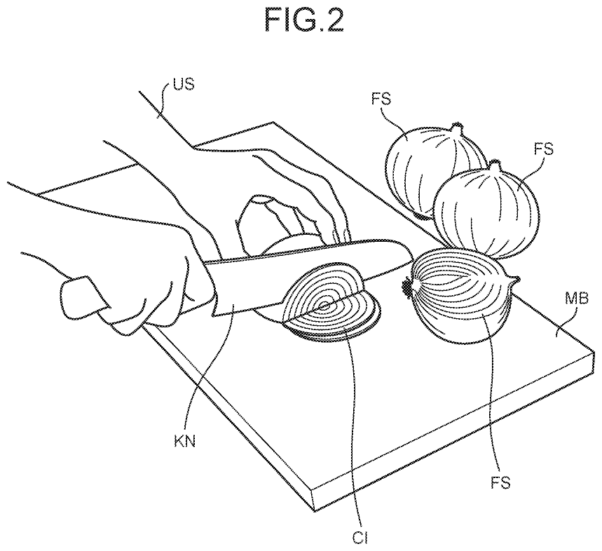

The information processing device IP acquires recipe information from a server SV via a router RT. The information processing device IP monitors cooking work by using a sensor unit SE. The information processing device IP corrects the recipe information on the basis of an internal state (moisture amount) of the foodstuff FS and a size of the cut ingredient CI detected by the sensor unit SE, and user input information input by user interface (UI) equipment IND. The information processing device IP corrects the recipe information as needed according to progress of cooking, and presents an appropriate cooking process to a cook US (see FIG. 2).

FIG. 2 is a view illustrating an example of a cooking scene.

A scene in which the foodstuff FS is cut with a knife KN is illustrated in FIG. 2. Cutting work is performed on a measurement board MB. The measurement board MB is used as a board on which the foodstuff FS is cut. The size and moisture amount of the cut foodstuff FS (cut ingredient CI) are automatically measured by the sensor unit SE. The measurement work is performed without hindering the cooking work in a natural flow of cutting and cooking the foodstuff FS. Thus, the cook US can concentrate on cooking without being conscious of the measurement work. The information processing device IP generates an optimal cooking process on the basis of a measurement result and presents the cooking process to the cook US.

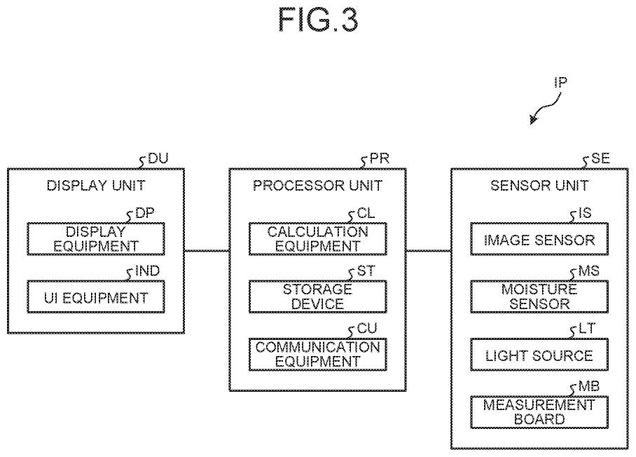

FIG. 3 is a functional block diagram of the information processing device IP.

The information processing device IP includes a sensor unit SE, a processor unit PR, and a display unit DU. The processor unit PR includes calculation equipment CL, a storage device ST, and communication equipment CU. The processor unit PR communicates with the sensor unit SE, the display unit DU, and the server SV by using the communication equipment CU. The processor unit PR stores various kinds of information acquired via the communication equipment CU into the storage device ST. The calculation equipment CL monitors the cooking work on the basis of the information acquired via the communication equipment CU and the information stored in the storage device ST, and generates assist information for assisting cooking.

For example, the processor unit PR monitors a state of the foodstuff FS by using sensor data acquired from the sensor unit SE. The processor unit PR optimizes the cooking process according to the state of the foodstuff cut in accordance with progress of cooking (cut ingredient CI). The processor unit PR presents information related to the optimized cooking process to the cook US as assistance information.

The assistance information is presented to the cook US via the display unit DU. The display unit DU includes display equipment DP and the UI equipment IND. The display equipment DP presents video information and audio information to the cook US. As the display equipment DP, a known display such as a liquid crystal display (LCD) or an organic light emitting diode (OLED) is used. The UI equipment IND receives an input of information from the cook US. The processor unit PR acquires user input information via the UI equipment IND. As the UI equipment IND, known input/output equipment such as a touch panel is used. Although the display equipment DP and the UI equipment IND are distinguished from each other in FIG. 3, these pieces of equipment can be integrally used as a tablet terminal.

The sensor unit SE includes one or more sensor functions. For example, the sensor unit SE includes an image sensor IS, a moisture sensor MS, a light source LT, and a measurement board MB. The image sensor IS photographs an uncut foodstuff FS and the cut foodstuff FS (cut ingredient CI) arranged on the measurement board MB. As the image sensor IS, for example, a known camera capable of capturing a visible light image is used. The moisture sensor MS measures a moisture amount of a cross section CS (cut surface) of the cut ingredient CI. As the moisture sensor MS, for example, a known moisture sensor capable of measuring a moisture amount in a non-contact manner, such as a near-infrared sensor is used.

The sensor unit SE monitors the cutting work performed on the measurement board MB. The sensor unit SE measures the size and the moisture amount of the foodstuff FS cut on the measurement board MB (cut ingredient CI) without interrupting the cutting work. The processor unit PR optimizes the cooking process on the basis of the measurement result of the sensor unit SE. The processor unit PR presents information of the optimized cooking process to the cook US via the display unit DU.

Although the image sensor IS and the moisture sensor MS are distinguished as logical functions in FIG. 3, these sensors do not necessarily include independent physical devices. One physical device may also have a plurality of sensor functions, or one sensor function may be realized by a combination of a plurality of physical devices.

### 2. Method of Measuring a Size of a Cut Ingredient

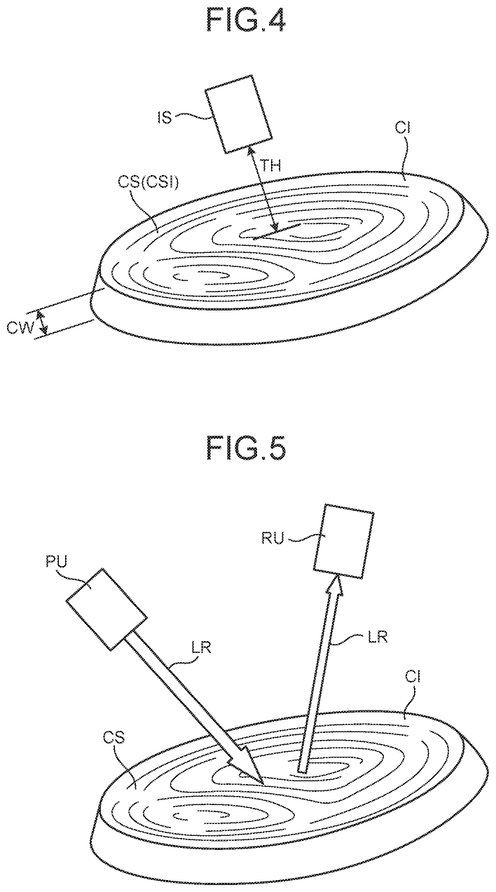

FIG. 4 is a view illustrating an example of a method of measuring a size of the cut ingredient CI.

The image sensor IS photographs the cut ingredient CI cut in accordance with the progress of cooking. The processor unit PR acquires information related to a cross-sectional image CSI and a cut width CW of the cut ingredient CI on the basis of an image captured by the image sensor IS. The processor unit PR detects a size (cross-sectional area and cut width CW) of the cut ingredient CI on the basis of the cross-sectional image CSI and the cut width CW of the cut ingredient CI. The processor unit PR calculates heating cooking time and a heating temperature of the cut ingredient CI on the basis of the size of the cut ingredient CI.

For example, the image sensor IS is attached to a back surface of the measurement board MB. The measurement board MB is configured as a colorless and transparent board that hardly absorbs visible light. The image sensor IS photographs the cut ingredient CI on the measurement board MB via the measurement board MB from a position separated for a thickness TH of the measurement board MB. The processor unit PR acquires the information related to the cross-sectional image CSI and the cut width CW of the cut ingredient CI from the image sensor IS attached to the back surface of the transparent measurement board MB. Since a distance between the image sensor IS and the cut ingredient CI is fixed, the size of the cut ingredient CI can be directly measured from the captured image by measurement of a relationship between a length in the captured image and an actual length by calibration in advance.

The image sensor IS captures not only the image of the cut ingredient CI but also that of the uncut foodstuff FS. The processor unit PR determines a kind of the foodstuff FS by using the image of the foodstuff FS before being cut into the cut ingredient CI. The kind of the foodstuff FS is determined by utilization of known object recognition technology. The processor unit PR calculates the heating cooking time and the heating temperature of the cut ingredient CI on the basis of the kind of the foodstuff FS.

### 3. Method of Measuring a Moisture Amount of a Cut Ingredient

FIG. 5 is a view illustrating an example of the method of measuring the moisture amount of the cut ingredient CI.

The moisture sensor MS includes a light projection unit PU and a light reception unit RU. The moisture sensor MS measures the moisture amount by using near infrared spectroscopy. The light projection unit PU projects light LR in a near infrared region that is an absorption wavelength region of water. The light reception unit RU receives the light LR reflected by the cross section CS of the cut ingredient CI. The moisture sensor MS calculates the moisture amount of the cut ingredient CI on the basis of an absorption amount of the light LR (difference between a light projection amount and a light reception amount), and outputs information of the calculated moisture amount to the processor unit PR.

A member that hardly absorbs near infrared rays is used as the measurement board MB. The processor unit PR detects the moisture amount of the cut ingredient CI on the basis of a sensing result of the cross section CS of the cut ingredient CI which result is from the moisture sensor MS. The processor unit PR calculates the heating cooking time and the heating temperature of the cut ingredient CI on the basis of the moisture amount of the cut ingredient CI.

For example, the moisture sensor MS is attached to the back surface of the measurement board MB. The moisture amount is measured in a natural flow of cutting the foodstuff FS. The moisture sensor MS senses the cross section CS of the cut ingredient CI cut in accordance with the progress of cooking without hindering the cutting work. Thus, the moisture sensor MS can measure an internal state of the foodstuff FS immediately before the heating cooking of the cut ingredient CI. The processor unit PR calculates the heating cooking time and the heating temperature of the cut ingredient CI on the basis of the moisture amount immediately before the heating cooking of the cut ingredient CI.

### 4. Detection of Gesture

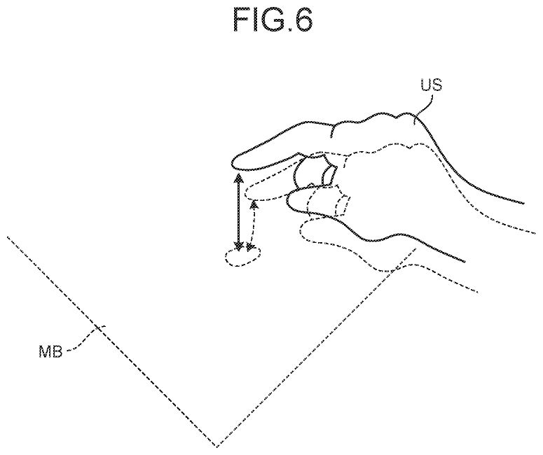

FIG. 6 is a view illustrating an example of gesture operation using the image sensor IS.

The cook US can perform gesture operation such as tapping on the measurement board MB. The image sensor IS captures video of the gesture performed on the measurement board MB and performs an output thereof to the processor unit PR. The processor unit PR detects the gesture of the cook US on the basis of the gesture video acquired from the image sensor IS. A relationship between the gesture and processing is stored in the storage device ST as gesture operation information. The processor unit PR collates the detected gesture with the gesture operation information and executes processing corresponding to the gesture. For example, when tap operation is detected in a predetermined region (such as an end portion) of the measurement board MB, the processor unit PR photographs the foodstuff FS or the cut ingredient CI on the measurement board MB by using the image sensor IS.

### 5. Processing Flow

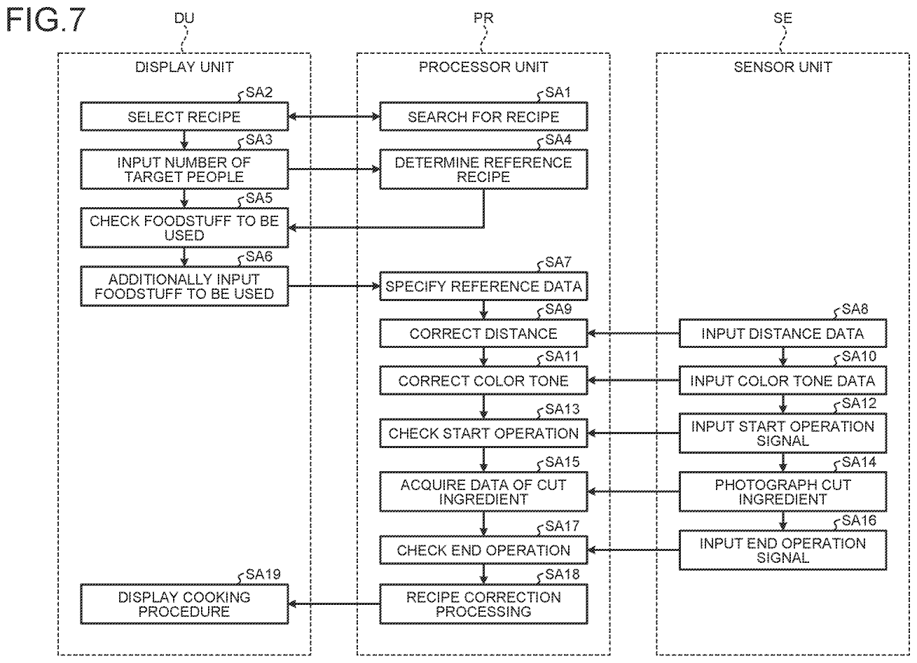

FIG. 7 is a view illustrating an example of a processing flow performed by the information processing device IP. Hereinafter, individual steps will be described according to a flow of the processing.

### [5-1. Specification of Reference Data]

The processor unit PR searches for a recipe of a dish desired by the cook US from the server SV on the basis of the user input information (Step SA1). By using the UI equipment IND, the cook US selects the recipe displayed on the display equipment DP (Step SA2). The cook US inputs, by using the UI equipment IND, the number of people (target number of people) to which the dish is provided (Step SA3). The processor unit PR downloads information related to the recipe selected by the cook US (recipe information) from the server SV, and determines, as a reference recipe, new recipe information in which an amount of the foodstuff FS and the like is corrected on the basis of the number of target people (Step SA4). The reference recipe includes information related to a kind, cut shape, and used amount of a representative foodstuff FS used for a dish, and heating cooking time and a heating temperature for each cooking process.

The processor unit PR checks the foodstuff to be used with the cook US via the display unit DU (Step SA5). In a case where it is desired to further add the foodstuff FS, the cook US performs input work related to the additional foodstuff by using the UI equipment IND (Step SA6). The processor unit PR downloads cooking data related to the additional foodstuff from the server SV and adds the cooking data to the reference recipe. As a result, the final recipe information including the additional foodstuff is specified as the reference data (Step SA7).

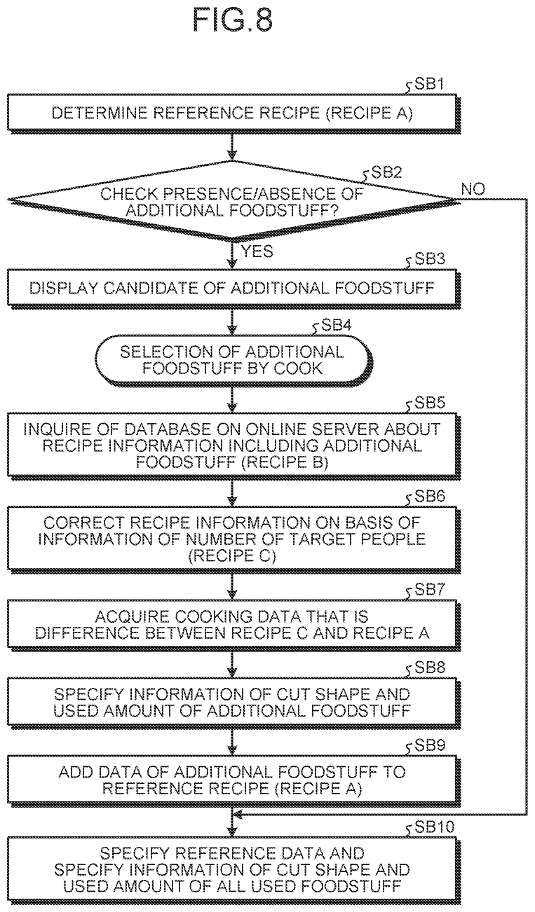

FIG. 8 is a view illustrating an example of a modification procedure of the reference recipe in a case where there is the additional foodstuff.

After determining the reference recipe (recipe A) (Step SB1), the processor unit PR checks whether there is the additional foodstuff with the cook US (Step SB2). In a case where there is the additional foodstuff (Step SB2: Yes), the processor unit PR displays candidates of the additional foodstuff on the display unit DU (Step SB3). The cook US selects a desired foodstuff from the displayed additional foodstuff list (Step SB4). In a case of adding a plurality of materials, foodstuffs can be added up to a selection upper limit.

The processor unit PR inquires of the server SV about recipe information including the additional foodstuff (recipe B) on the basis of information of the additional foodstuff (Step SB5). The processor unit PR corrects the used amounts of the foodstuffs in the recipe B on the basis of the information of the target number of people which information is used when the reference recipe is determined, and generates new recipe information (recipe C) (Step SB6). The processor unit PR downloads a difference between the recipe C to which the additional foodstuff is added and the reference recipe (recipe A) from the server SV as cooking data (Step SB7). Information of a cut shape and a used amount of the additional foodstuff is specified on the basis of the downloaded cooking data (Step SB8).

The processor unit PR adds the downloaded cooking data to the reference recipe (Step SB9). As a result, the reference data is specified (Step SB10). The reference data includes information related to the kind, cut shape, and used amount of all the foodstuffs FS used for the dish, and the heating cooking time and heating temperature for each cooking process.

### [5-2. Data Acquisition for Correction Processing]

Returning to FIG. 7, the cutting work is repeatedly performed on the measurement board MB. When the measurement board MB is continuously used for a long period, a surface shape of the measurement board MB changes or discoloration is generated, and a sensing result may be affected. Thus, before cooking, distance data and color tone data of the image sensor IS are corrected (Step SA8 to SA11).

For example, the cook US installs, on the surface of the measurement board MB, a sheet on which scales are printed at equal intervals on lines drawn in a cross shape. The image sensor IS photographs the scales of the sheet from a side of the back surface of the measurement board MB and performs an output thereof as the distance data (Step SA8). When the measurement board MB is uneven, the intervals between the scales vary. The processor unit PR acquires distribution of the intervals of the scales appearing in the captured image as calibration data of the image sensor IS. The processor unit PR uses the calibration data of the image sensor IS as reference information for calculating the size of the cut ingredient CI (Step SA9).

The color tone data is corrected by utilization of, for example, a color chart or a gray card used for color tone correction of video equipment. The image sensor IS outputs the captured image of the color chart or the gray card as the color tone data (Step SA10). The processor unit PR acquires a gap between a color appearing in the captured image and an actual color as color tone correction data. The processor unit PR performs color tone correction by using the color tone correction data (Step SA11). By performing the color tone correction of the image sensor IS, the color of the foodstuff FS and the cut ingredient CI can be correctly recognized. The processor PR determines the kind of the foodstuff FS on the basis of the captured image of the uncut foodstuff FS. When the color can be correctly recognized, the determination accuracy of the foodstuff FS is also enhanced.

### [5-3. Acquisition of Data of a Cut Ingredient]

When the distance correction and the color tone correction are over, the cook US starts cooking. The image sensor IS inputs a start operation signal to the processor unit PR on the basis of start operation of the cook US (Step SA12). The processor unit PR checks the start operation on the basis of the start operation signal (Step SA13).

After the start of cooking, the cutting work and the measurement work of the foodstuff FS are performed in parallel (Step SA14 to SA15). The measurement is started and ended by the cook US. For example, the measurement is started when the lower right of the measurement board MB is touched for two seconds, and the measurement is ended when the upper left of the measurement board MB is touched for two seconds.

The image sensor IS inputs an end operation signal to the processor unit PR on the basis of end operation of the cook US (Step SA16). The processor unit PR checks the end operation on the basis of the end operation signal (Step SA17).

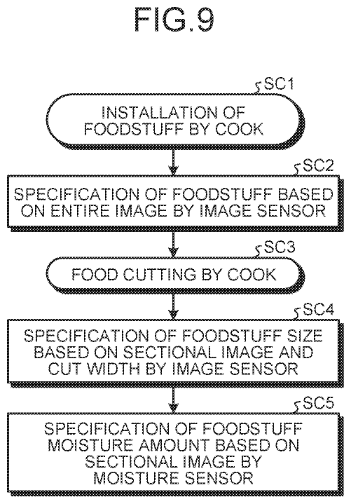

FIG. 9 is a view illustrating a flow of the measurement work by the sensor unit SE.

After performing the start operation, the cook US installs the foodstuff FS on the measurement board MB (Step SC1). The cook US captures an entire image of the foodstuff FS by the image sensor IS before cutting the foodstuff FS. The processor unit PR applies the captured image of the entire image to an identification model for object recognition which model is generated in advance by machine learning, and specifies the kind of the foodstuff FS on the measurement board MB (Step SC2). The identification model is generated by learning of feature amounts of positive example data of various foodstuffs FS.

When the specification of the foodstuff FS is over, the cook US starts the cutting work of the foodstuff FS (Step SC3). The processor unit PR detects the cut width CW of the foodstuff FS on the basis of the video of the cutting work captured by the image sensor IS. The processor unit PR detects a cross-sectional area of the cut ingredient CI on the basis of the cross-sectional image CSI captured when the cross section of the cut ingredient CI comes into contact with the measurement board MB. The processor unit PR specifies the size of the cut ingredient CI on the basis of the cross-sectional area and the cut width CW of the cut ingredient CI (Step SC4).

Note that in a case where the cross-sectional image CSI is unclear, the processor unit PR specifies a cutting method by applying the identification model generated in advance by machine learning, and specifies the size of the cut ingredient CI. This identification model is generated by learning of the feature amounts of the positive example data of the cut ingredient CI generated by cutting of the foodstuff FS by various cutting methods.

The moisture sensor MS inputs data measured when the cross section of the cut ingredient CI comes into contact with the measurement board MB to the processor unit PR. The processor unit PR compares the data acquired from the moisture sensor MS with a data table in which the moisture amount of each of the foodstuffs FS is recorded. As a result, the processor unit PR specifies the moisture amount of the cut ingredient CI (Step SC5).

### [5-4. Display of a Cooking Procedure]

Returning to FIG. 7, the processor unit PR applies the size and the moisture amount of the cut ingredient CI to an estimation model, and estimates the optimal heating cooking time and heating temperature (heating power) of the cut ingredient CI. The processor unit PR corrects the information related to the heating cooking time and the heating temperature of the reference data on the basis of an estimation result (Step SA18). As the estimation model, for example, a learned neural network acquired by machine learning of a relationship between the kind, size, and moisture amount of the cut ingredient CI and the heating cooking time and heating temperature for each cooking process is used.

The processor unit PR acquires the optimal heating cooking time and heating temperature of the cut ingredient CI by inputting, to the estimation model, information of the kind, size, and moisture amount of the cut ingredient CI which information is detected by utilization of the sensor information. The processor unit PR displays the acquired heating cooking time and heating temperature on display unit DU as an optimized cooking procedure (Step SA19).

### 6. Hardware Configuration Example

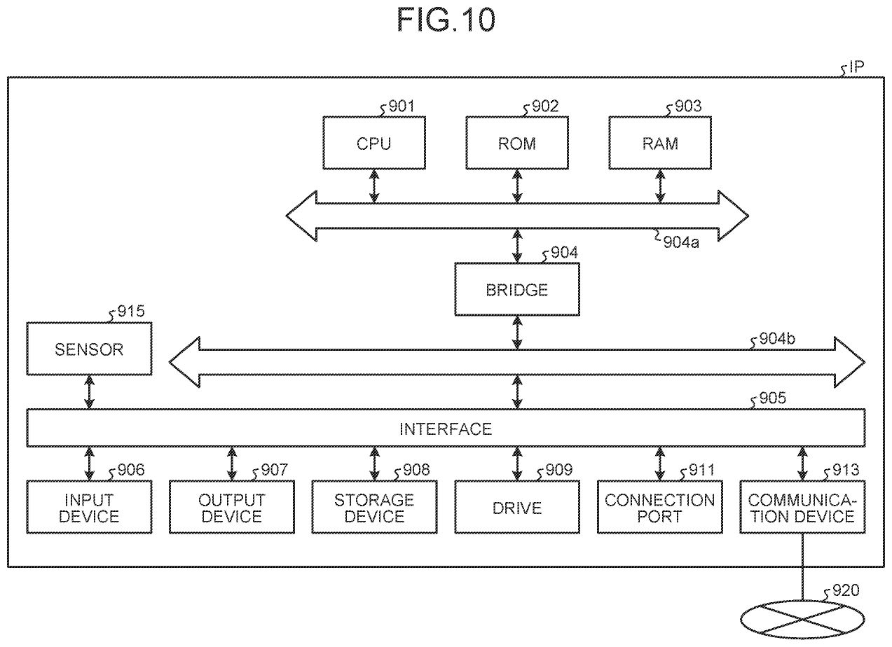

FIG. 10 is a view illustrating a hardware configuration example of the information processing device IP.

The information processing device IP includes a central processing unit (CPU) 901, a read only memory (ROM) 902, a random access memory (RAM) 903, and a host bus 904a. In addition, the information processing device IP includes a bridge 904, an external bus 904b, an interface 905, an input device 906, an output device 907, a storage device 908, a drive 909, a connection port 911, a communication device 913, and a sensor 915. The information processing device IP may include a processing circuit such as a DSP or an ASIC instead of or in addition to the CPU 901.

The CPU 901 functions as an arithmetic processing unit and a control device, and controls overall operations in the information processing device IP according to various programs. Also, the CPU 901 may be a microprocessor. The ROM 902 stores programs, operation parameters, and the like used by the CPU 901. The RAM 903 temporarily stores the programs used in execution of the CPU 901, parameters that appropriately change in the execution, and the like. The CPU 901 can form, for example, the processor unit PR.

The CPU 901, the ROM 902, and the RAM 903 are connected to each other by the host bus 904a including a CPU bus and the like. The host bus 904a is connected to the external bus 904b such as a peripheral component interconnect/interface (PCI) bus via the bridge 904. Note that it is not necessary to configure the host bus 904a, the bridge 904, and the external bus 904b separately, and these functions may be mounted on one bus.

The input device 906 is realized by, for example, a device to which information is input by a user, such as a mouse, a keyboard, a touch panel, a button, a microphone, a switch, and a lever. Furthermore, the input device 906 may be, for example, a remote control device using infrared rays or other radio waves, or may be external connection equipment such as a mobile phone or a PDA corresponding to the operation of the information processing device IP. Furthermore, the input device 906 may include, for example, an input control circuit or the like that generates an input signal on the basis of the information input by the user by utilization of the above input units, and that performs an output thereof to the CPU 901. By operating the input device 906, the user of the information processing device IP can input various kinds of data to the information processing device IP or instruct the information processing device IP to perform a processing operation. The input device 906 may form, for example, the UI equipment IND.

The output device 907 includes a device capable of visually or aurally notifying the user of the acquired information. Examples of such a device include a display device such as a CRT display device, a liquid crystal display device, a plasma display device, an EL display device, and a lamp, an audio output device such as a speaker and a headphone, and a printer device. The output device 907 outputs, for example, results acquired by various kinds of processing performed by the information processing device IP. Specifically, the display device visually displays the results, which are acquired by the various kinds of processing performed by the information processing device IP, in various formats such as text, an image, a table, and a graph. On the other hand, the audio output device converts an audio signal including reproduced voice data, acoustic data, or the like into an analog signal and performs an aural output thereof. The output device 907 may form, for example, the display equipment DP.

The storage device 908 is a device that is for data storage and that is formed as an example of a storage unit of the information processing device IP. The storage device 908 is realized, for example, by a magnetic storage unit device such as an HDD, a semiconductor storage device, an optical storage device, a magneto-optical storage device, or the like. The storage device 908 may include a storage medium, a recording device that records data into the storage medium, a reading device that reads the data from the storage medium, a deletion device that deletes the data recorded in the storage medium, and the like. The storage device 908 stores programs executed by the CPU 901, various kinds of data, various kinds of data acquired from the outside, and the like. The storage device 908 can form, for example, the storage device ST.

The drive 909 is a reader/writer for a storage medium, and is built in or externally attached to the information processing device IP. The drive 909 reads information recorded in a mounted removable storage medium such as a magnetic disk, optical disk, magneto-optical disk, or semiconductor memory, and performs an output thereof to the RAM 903. Also, the drive 909 can write information into the removable storage medium.

The connection port 911 is an interface connected to external equipment, and is a connection port to external equipment capable of transmitting data by, for example, a universal serial bus (USB).

The communication device 913 is, for example, a communication interface formed of a communication device or the like for connection to a network 920. The communication device 913 is, for example, a communication card for a wired or wireless local area network (LAN), long term evolution (LTE), Bluetooth (registered trademark), or a wireless USB (WUSB), or the like. Also, the communication device 913 may be a router for optical communication, a router for an asymmetric digital subscriber line (ADSL), a modem for various kinds of communication, or the like. On the basis of a predetermined protocol such as TCP/IP, the communication device 913 can transmit/receive a signal or the like to/from the Internet or another communication equipment, for example. The communication device 913 can form, for example, communication equipment CU.

The sensor 915 is, for example, various sensors such as the image sensor IS and the moisture sensor MS. The sensor 915 acquires information related to a state of an object to be cooked and information related to surrounding environment of the cooking support system CSP, such as brightness and noise around the information processing device IP. The sensor 915 can form, for example, the sensor unit SE.

Note that the network 920 is a wired or wireless transmission path of information transmitted from a device connected to the network 920. For example, the network 920 may include a public network such as the Internet, a telephone network, or a satellite communication network, various local area networks (LAN) including Ethernet (registered trademark), a wide area network (WAN), and the like. Also, the network 920 may include a dedicated network such as the Internet protocol-virtual private network (IP-VPN).

### 7. Modification Example

### [7-1. Addition of a Weight Sensor, a Hardness Sensor, and a Temperature Sensor]

The sensor unit SE may have a sensor function other than the image sensor IS and the moisture sensor MS. For example, the sensor unit SE may include a weight sensor that measures a weight of the cut ingredient CI, a hardness sensor that measures texture of the cut ingredient CI, and a temperature sensor that measures a surface temperature of the cut ingredient CI. These sensors may be built in a cooking utensil such as the measurement board MB. Measurement data by these sensors is also transmitted to the processor unit PR as time series data.

For example, in a case where the sensor unit SE includes the weight sensor, the processor unit PR calculates the heating cooking time and the heating temperature on the basis of a sensing result of the weight of the cut ingredient CI. In a case where the sensor unit SE includes the temperature sensor, it is possible to predict a temperature decrease due to addition of the cut ingredient CI during the heating cooking.

### [7-2. Transfer of Cooking Process Data to a Cooking Equipment]

The cooking support system CSP can include a cooking equipment that traces and reproduces the cooking process calculated by the processor unit PR. The cooking equipment acquires information related to the optimal heating cooking time and heating temperature calculated by the processor unit PR, and automatically heats and cooks the cut ingredient CI put in the cooking equipment. In this case, the cook US can perform cooking without controlling the heating temperature or checking the heating cooking time.

### 8. Effect

The information processing device IP includes a processor unit PR. The processor unit PR detects the moisture amount of the cut ingredient CI on the basis of a sensing result of the cross section of the cut ingredient CI cut in accordance with the progress of cooking. The processor unit PR calculates the heating cooking time and the heating temperature of the cut ingredient CI on the basis of the moisture amount of the cut ingredient CI. In the information processing method of the present disclosure, processing of the information processing device IP is executed by a computer. The program of the present disclosure causes a computer to realize processing of the information processing device IP.

According to this configuration, the cooking method is appropriately adjusted according to the moisture amount of the foodstuff FS (internal state of the foodstuff FS). Measurement of the moisture amount is performed as part of the cooking work of cutting the foodstuff FS and acquiring the cut ingredient CI. Since the cooking work is not interrupted in order to measure the moisture amount, cooking is smoothly performed. Conventionally, the moisture amount has been estimated by tasting of a food piece by the cook in addition to the determination of the appearance. From a viewpoint of avoiding an adverse effect on hygiene, it is very useful to measure the moisture amount in a natural operation of cutting the foodstuff FS.

The processor unit PR calculates the heating cooking time and the heating temperature on the basis of the moisture amount immediately before heating cooking of the cut ingredient CI.

According to this configuration, an appropriate cooking method corresponding to a state of the foodstuff FS immediately before the heating cooking is presented.

The processor unit PR detects the size of the cut ingredient CI on the basis of the cross-sectional image CSI and the cut width CW of the cut ingredient CI. The processor unit PR calculates the heating cooking time and the heating temperature on the basis of the detected size of the cut ingredient CI.

According to this configuration, the cooking method is appropriately adjusted according to the size of the cut ingredient CI.

The processor unit PR acquires information related to the cross-sectional image CSI and the cut width CW of the cut ingredient CI cut on the measurement board MB from the image sensor IS attached to the back surface of the transparent measurement board MB.

According to this configuration, photographing of the cut ingredient CI is not hindered by the cutting work of the foodstuff FS. Thus, the cutting work and the measurement work of the cut ingredient CI are smoothly performed.

The processor unit PR uses the calibration data of the image sensor IS as reference information for calculating the size of the cut ingredient CI.

According to this configuration, it is possible to directly acquire information related to the size of the cut ingredient CI from the captured image without separately preparing a reference substance serving as a reference of the size.

The processor unit PR determines a kind of the foodstuff FS by using the image of the foodstuff FS before being cut into the cut ingredient CI. The processor unit PR calculates the heating cooking time and the heating temperature on the basis of the determined kind of the foodstuff FS.

According to this configuration, the cooking method is appropriately adjusted on the basis of information of both the kind and the size of the cut ingredient CI.

The processor unit PR detects the gesture of the cook US on the basis of the gesture video acquired from the image sensor IS, and executes processing corresponding to the gesture.

According to this configuration, the instruction by the gesture can be smoothly performed in the flow of the cooking operation without interrupting the cooking work.

The processor unit PR calculates the heating cooking time and the heating temperature on the basis of the sensing result of the weight of the cut ingredient CI.

According to this configuration, the cooking method is appropriately adjusted according to the weight of the cut ingredient CI.

The cooking support system CSP includes the image sensor IS, the moisture sensor MS, and the processor unit PR. The image sensor IS photographs the cut ingredient CI cut in accordance with the progress of cooking. The moisture sensor MS measures the moisture amount of the cross section of the cut ingredient CI. The processor unit PR calculates the size of the cut ingredient CI on the basis of the captured image of the cut ingredient CI. The processor unit PR calculates the heating cooking time and the heating temperature of the cut ingredient CI on the basis of the size and the moisture amount of the cut ingredient CI.

According to this configuration, the cooking method is appropriately adjusted according to the moisture amount of the foodstuff FS (internal state of the foodstuff FS). Measurement of the moisture amount is performed as part of the cooking work of cutting the foodstuff FS and acquiring the cut ingredient CI. Since the cooking work is not interrupted in order to measure the moisture amount, cooking is smoothly performed.

Note that the effects described in the present description are merely examples and are not limitations, and there may be another effect.

### [Supplementary Note]

Note that the present technology can also have the following configurations.

(1)

An information processing device comprising: a processor unit that detects a moisture amount of a cut ingredient on a basis of a sensing result of a cross section of the cut ingredient cut in accordance with progress of cooking, and calculates heating cooking time and a heating temperature of the cut ingredient on a basis of the moisture amount of the cut ingredient.

(2)

The information processing device according to (1), wherein
the processor unit calculates the heating cooking time and the heating temperature on a basis of the moisture amount immediately before the cut ingredient is heated and cooked.

(3)

The information processing device according to (2), wherein
the processor unit detects a size of the cut ingredient on a basis of a cross-sectional image and a cut width of the cut ingredient, and calculates the heating cooking time and the heating temperature on a basis of the size of the cut ingredient.

(4)

The information processing device according to (3), wherein
the processor unit acquires, from an image sensor attached to a back surface of a transparent measurement board, information related to the cross-sectional image and the cut width of the cut ingredient cut on the measurement board.

(5)

The information processing device according to (4), wherein
the processor unit uses calibration data of the image sensor as reference information to calculate the size of the cut ingredient.

(6)

The information processing device according to (4) or (5), wherein
the processor unit determines a kind of a foodstuff by using an image of the foodstuff before being cut into the cut ingredient, and calculates the heating cooking time and the heating temperature on a basis of the kind of the foodstuff.

(7)

The information processing device according to any one of (4) to (6), wherein
the processor unit detects gesture of a cook on a basis of a gesture video acquired from the image sensor and executes processing corresponding to the gesture.

(8)

The information processing device according to any one of (1) to (7), wherein
the processor unit calculates the heating cooking time and the heating temperature on a basis of a sensing result of a weight of the cut ingredient.

(9)

An information processing method executed by a computer, the method comprising:
detecting a moisture amount of a cut ingredient on a basis of a sensing result of a cross section of the cut ingredient cut in accordance with progress of cooking; and
calculating heating cooking time and a heating temperature of the cut ingredient on a basis of the moisture amount of the cut ingredient.

(10)

A program causing a computer to realize
detecting a moisture amount of a cut ingredient on a basis of a sensing result of a cross section of the cut ingredient cut in accordance with progress of cooking, and
calculating heating cooking time and a heating temperature of the cut ingredient on a basis of the moisture amount of the cut ingredient.

(11)

A cooking support system including:
an image sensor that photographs a cut ingredient cut in accordance with progress of cooking;
a moisture sensor that measures a moisture amount of a cross section of the cut ingredient; and
a processor unit that calculates a size of the cut ingredient on the basis of a captured image of the cut ingredient, and calculates heating cooking time and a heating temperature of the cut ingredient on the basis of the size and the moisture amount of the cut ingredient.

(12)

The cooking support system according to (11), further including
a measuring board used as a board on which a foodstuff is cut, in which
the image sensor is installed on a back surface of the measurement board and photographs the cut ingredient on the measurement board via the measurement board.

### REFERENCE SIGNS LIST

CI CUT INGREDIENT
CS CROSS SECTION
CSI CROSS-SECTIONAL IMAGE
CW CUT WIDTH
FS FOODSTUFF
IP INFORMATION PROCESSING DEVICE
IS IMAGE SENSOR
MB MEASUREMENT BOARD
PR PROCESSOR UNIT
US COOK

1. An information processing device comprising: a processor unit that detects a moisture amount of a cut ingredient on a basis of a sensing result of a cross section of the cut ingredient cut in accordance with progress of cooking, and calculates heating cooking time and a heating temperature of the cut ingredient on a basis of the moisture amount of the cut ingredient.

2. The information processing device according to claim 1, wherein
the processor unit calculates the heating cooking time and the heating temperature on a basis of the moisture amount immediately before the cut ingredient is heated and cooked.

3. The information processing device according to claim 2, wherein
the processor unit detects a size of the cut ingredient on a basis of a cross-sectional image and a cut width of the cut ingredient, and calculates the heating cooking time and the heating temperature on a basis of the size of the cut ingredient.

4. The information processing device according to claim 3, wherein
the processor unit acquires, from an image sensor attached to a back surface of a transparent measurement board, information related to the cross-sectional image and the cut width of the cut ingredient cut on the measurement board.

5. The information processing device according to claim 4, wherein
the processor unit uses calibration data of the image sensor as reference information to calculate the size of the cut ingredient.

6. The information processing device according to claim 4, wherein
the processor unit determines a kind of a foodstuff by using an image of the foodstuff before being cut into the cut ingredient, and calculates the heating cooking time and the heating temperature on a basis of the kind of the foodstuff.

7. The information processing device according to claim 4, wherein
the processor unit detects gesture of a cook on a basis of a gesture video acquired from the image sensor and executes processing corresponding to the gesture.

8. The information processing device according to claim 1, wherein
the processor unit calculates the heating cooking time and the heating temperature on a basis of a sensing result of a weight of the cut ingredient.

9. An information processing method executed by a computer, the method comprising:
detecting a moisture amount of a cut ingredient on a basis of a sensing result of a cross section of the cut ingredient cut in accordance with progress of cooking; and
calculating heating cooking time and a heating temperature of the cut ingredient on a basis of the moisture amount of the cut ingredient.

10. A program causing a computer to realize
detecting a moisture amount of a cut ingredient on a basis of a sensing result of a cross section of the cut ingredient cut in accordance with progress of cooking, and
calculating heating cooking time and a heating temperature of the cut ingredient on a basis of the moisture amount of the cut ingredient.
